(19) United States
(12) Patent Application Publication
(10) Pub. No.: US 2015/0327913 A1
(43) Pub. Date: Nov. 19, 2015

(54) SURGICAL INSTRUMENT WITH EXTENDIBLE MONOPOLAR ELEMENT

(71) Applicant: COVIDIEN LP, MANSFIELD, MA (US)

(72) Inventor: GLENN A. HORNER, BOULDER, CO (US)

(21) Appl. No.: 14/564,631

(22) Filed: Dec. 9, 2014

Related U.S. Application Data
(60) Provisional application No. 61/993,396, filed on May 15, 2014.

(51) Int. Cl. A61B 18/14; A61B 18/12

(52) U.S. Cl. CPC A61B 18/1445; A61B 18/1206; A61B 2018/1253; A61B 2018/00708

(57) ABSTRACT

The present disclosure relates to an electrosurgical instrument that includes a housing having a shaft that extends therefrom and an end effector assembly attached at the distal end of the shaft. The end effector assembly includes a monopolar element slidably disposed therein that is configured to move between a retracted position and an extended configuration. A monopolar activation switch is disposed within the housing and is configured to supply energy to the monopolar element upon actuation thereof. An actuating sleeve is moveable relative to the housing and is operably coupled to the monopolar element. The actuating sleeve is movable between a first position wherein the monopolar element is disposed in the retracted configuration and actuation of the monopolar activation switch is impeded and a second position wherein the monopolar element is disposed in the extended configuration and actuation of the monopolar activation switch is unimpeded.

### CROSS REFERENCE TO RELATED APPLICATION

The present application claims the benefit of and priority to U.S. Provisional Application Ser. No. 61/993,396, filed on May 15, 2014, the entire contents of which are incorporated herein by reference.

### BACKGROUND

1. Technical Field

The present disclosure relates to the use of medical instruments. More particularly, the present disclosure is directed to bipolar devices with selectively extendible monopolar elements.

2. Background of the Related Art

A surgical forceps is a plier-like instrument which relies on mechanical action between its jaw members to grasp, clamp, and constrict tissue. Energy-based surgical forceps utilize both mechanical clamping action and energy, e.g., RF energy, ultrasonic energy, microwave energy, thermal energy, light energy, etc., to affect hemostasis by heating tissue and blood vessels to coagulate and/or cauterize tissue. Certain surgical procedures require more than simply coagulating/cauterizing tissue and rely on the unique combination of clamping pressure, precise energy control, and gap distance (i.e., the distance between opposing jaw members when closed about tissue) to “seal” tissue.

Typically, once tissue is treated, e.g., sealed, the surgeon has to accurately sever the tissue along the newly formed tissue seal. Accordingly, many surgical forceps have been designed which incorporate a knife or blade member that effectively severs the tissue after forming a tissue seal.

In some cases, the user may desire to use an electrosurgical instrument with a bipolar arrangement to treat tissue in a certain fashion, e.g., bipolar jaw members, and a different electrosurgical instrument to treat tissue in a monopolar fashion, e.g., dissect tissue. Electrosurgical instruments that include both monopolar and bipolar functionality have been developed to avoid unnecessarily substituting instruments within the surgical cavity to perform different surgical functions. Some of these instruments may be cumbersome, difficult to manufacture or lack adequate features to safely switch between bipolar and monopolar modes.

### SUMMARY

As shown in the drawings and described throughout the following description, as is traditional when referring to relative positioning on a surgical instrument, the term “proximal” refers to the end of the apparatus that is closer to the user and the term “distal” refers to the end of the apparatus that is farther away from the user. The term “clinician” refers to any medical professional (e.g., doctor, surgeon, nurse, or the like) performing a medical procedure involving the use of embodiments described herein.

In at least one aspect of the present disclosure, an electrosurgical instrument includes an end effector assembly having a monopolar element moveably disposed therein. Although any desired end effector assembly may be employed in conjunction with the monopolar element, for the sake of brevity, the description of the electrosurgical instrument will be limited herein to an electrosurgical forceps.

In one aspect of the present disclosure, an electrosurgical instrument includes a housing having a shaft that extends therefrom and an end effector assembly attached at the distal end of the shaft. The end effector assembly includes a monopolar element moveably or slidably disposed therein that is configured to move between a retracted position and an extended configuration. A monopolar activation switch is disposed within the housing and is configured to supply energy to the monopolar element upon actuation thereof.

An actuating sleeve is movable relative to the housing and is operably coupled to the monopolar element. The actuating sleeve is movable between a first position wherein the monopolar element is disposed in the retracted configuration and actuation of the monopolar activation switch is impeded and a second position wherein the monopolar element is disposed in the extended configuration and actuation of the monopolar activation switch is unimpeded.

In one aspect, the actuation sleeve covers the monopolar activation switch when disposed in the first position. In another aspect, the actuation sleeve exposes the monopolar activation switch for actuation thereof when disposed in the second position. In still another aspect, energy is prevented from flowing to the monopolar element unless the actuating sleeve is proximate to or disposed in the second position. In other aspects, electrosurgical energy may be prevented from flowing to the monopolar element when the actuating sleeve is proximate to or disposed in the first position as an additional safety feature.

In yet another aspect, the end effector assembly includes first and second jaw members at least one of which is movable relative to the other between a spaced-apart position and an approximated position for grasping tissue therebetween. Each jaw member includes an electrically-conductive tissue-contacting surface adapted to connect to a source of electrosurgical energy to treat tissue grasped between the jaw members. In one aspect, electrosurgical energy may be prevented from flowing to the first and second jaw members unless the actuating sleeve is proximate to or disposed in the first position. In other aspects, electrosurgical energy may be prevented from flowing to the first and second jaw members when the actuating sleeve is proximate to or disposed in the second position. In still other aspects, either or both jaw members may be used as a return path during monopolar activation of the monopolar element.

In aspects of the present disclosure, the configuration of the monopolar element may be in the shape of a hook, loop, blade, partial loop, straight, tapered, flared, ball, and needle. Other known geometric configurations for monopolar elements are also contemplated.

In yet another aspect of the present disclosure, an activation switch is disposed on the housing and is configured to supply electrosurgical energy to the end effector assembly or the jaw members of the end effector assembly. Movement of the actuating sleeve from the first to second positions simultaneously places the monopolar activation switch in-circuit for selective energization of the monopolar element and places the activation switch out-of-circuit to impede the supply of electrosurgical energy to the end effector assembly.

The present disclosure also relates to a method for treating tissue and includes providing a surgical instrument having a housing with a shaft that extends therefrom and an end effector assembly attached at the distal end of the shaft. The end effector assembly includes a monopolar element slidably disposed therein that is configured to move between a retracted position and an extended configuration. The method also provides a monopolar activation switch disposed within the housing that is configured to supply energy to the monopolar element upon actuation thereof and an actuating sleeve is operably coupled to the monopolar element.

The method also includes moving the actuating sleeve relative to the housing between a first position wherein the monopolar element is disposed in the retracted configuration and actuation of the monopolar activation switch is impeded and a second position wherein the monopolar element is disposed in the extended configuration and actuation of the monopolar activation switch is unimpeded.

The surgical instrument may include an activation switch configured to energize the end effector assembly for treating tissue in a bipolar manner and movement of the actuating sleeve from the first to second positions may simultaneously place the monopolar activation switch in-circuit for selective energization of the monopolar element and place the activation switch out-of-circuit to impede the supply of electrosurgical energy to the end effector assembly.

The present disclosure may also relate to a method for performing a surgical procedure including moving or sliding an actuating sleeve relative to a housing of a surgical instrument to simultaneously expose a monopolar activation switch and extend a monopolar element from an end effector assembly disposed at a distal end of a shaft extending from the housing. The method may also include actuating the monopolar activation switch to energize the monopolar element to treat tissue. The moving or sliding of the actuating sleeve may also deactivate an activation switch configured to energize the end effector assembly or the jaw members.

In another aspect of the present disclosure, a safety mechanism is included (e.g., a mechanical or electrical) whereby movement of the actuating sleeve completes a circuit which, upon actuation of the respective switches allows electrosurgical energy to flow to the monopolar element when the actuating sleeve is disposed in the second position or to the jaw members when the actuating sleeve is disposed in the first position. A sensor may be included that senses when the actuating sleeve is positioned sufficiently close to the first or second position such that the sensor allows electrosurgical energy to flow to the jaw members or monopolar element, respectively.

### BRIEF DESCRIPTION OF THE DRAWINGS

The above and other aspects, features, and advantages of the present disclosure will become more apparent in view of the following detailed description when taken in conjunction with the accompanying drawings in which:

FIG. 1A is a front, perspective view of a medical device having a housing including an elongated shaft extending therefrom with an end effector assembly attached to a distal end thereof in accordance with one embodiment of the present disclosure;

FIG. 1B is a rear, perspective view of the medical device of FIG. 1A showing part of the housing removed;

FIG. 1C is an enlarged, side view of a handle of the medical device of FIG. 1B shown in a first, unactuated position relative to the housing;

FIG. 1D is an enlarged, side view of the handle of the medical device of FIG. 1B shown in a second, actuated position relative to the housing;

FIG. 2 is a side view of the device of FIG. 1A showing an actuating sleeve in a retracted configuration;

FIG. 3 is a side view of the device of FIG. 1A showing the actuating sleeve in an extended configuration relative to the housing thereby exposing a monopolar element;

FIG. 4 is a partial, cross-sectional, side view of the actuating sleeve in the retracted configuration with the monopolar element shown housed within the end effector assembly; and

FIG. 5 is a partial, cross-sectional, side view of the actuating sleeve in the extended configuration with the monopolar element shown extending from the end effector assembly.

### DETAILED DESCRIPTION

Particular embodiments of the present disclosure are described hereinbelow with reference to the accompanying drawings; however, the disclosed embodiments are merely examples of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure. Like reference numerals may refer to similar or identical elements throughout the description of the figures.

In at least one aspect of the present disclosure, an electrosurgical instrument is provided that includes an actuating sleeve that is selectively translatable to both extend a monopolar element from a housed orientation while simultaneously exposing an activating switch for energizing the monopolar element. Although any desired electrosurgical instrument may be employed in conjunction with the monopolar element, for the sake of brevity, the description of the electrosurgical instrument herein will be limited to an electrosurgical forceps for use with endoscopic surgical procedures. In other embodiments, the electrosurgical instrument may be any type of instrument, including, but not limited to, open electrosurgical forceps, scissors, and the like. Different electrical and mechanical connections and considerations may apply to each particular type of device, however, the aspects and features of the present disclosure remain generally consistent regardless of the particular device used.

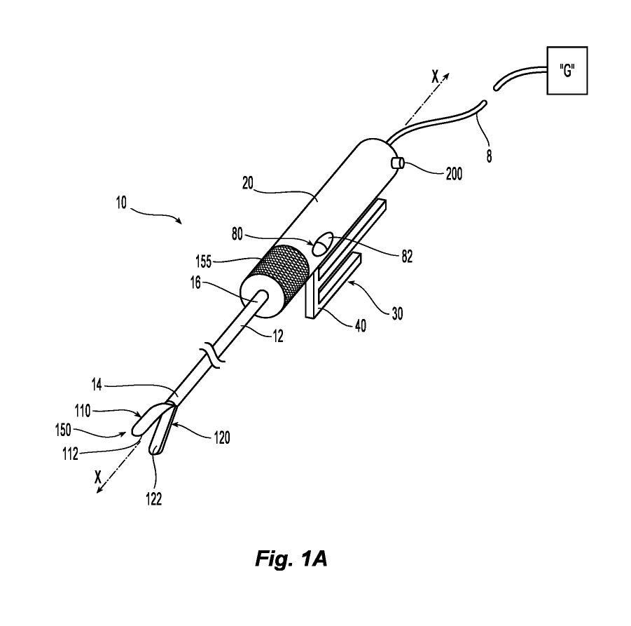

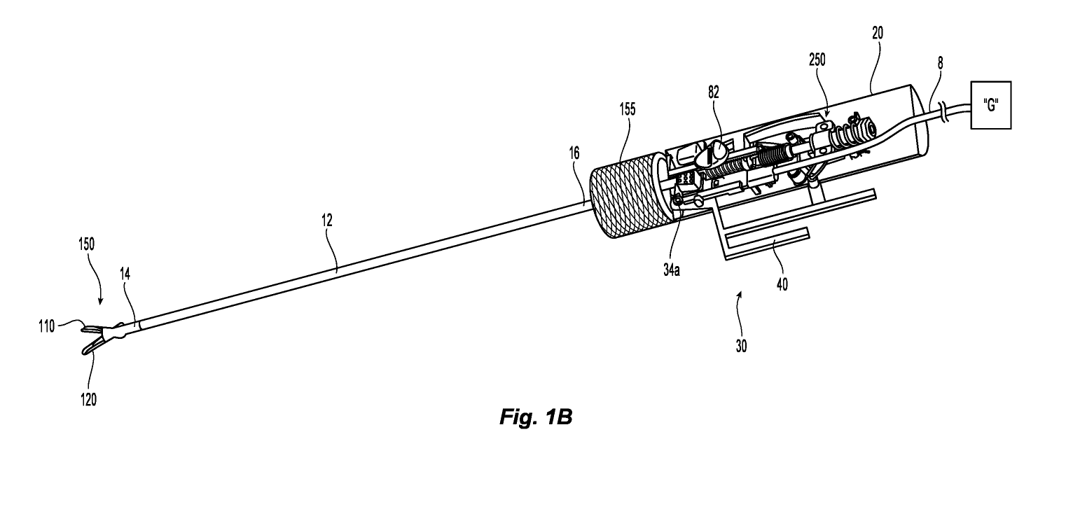

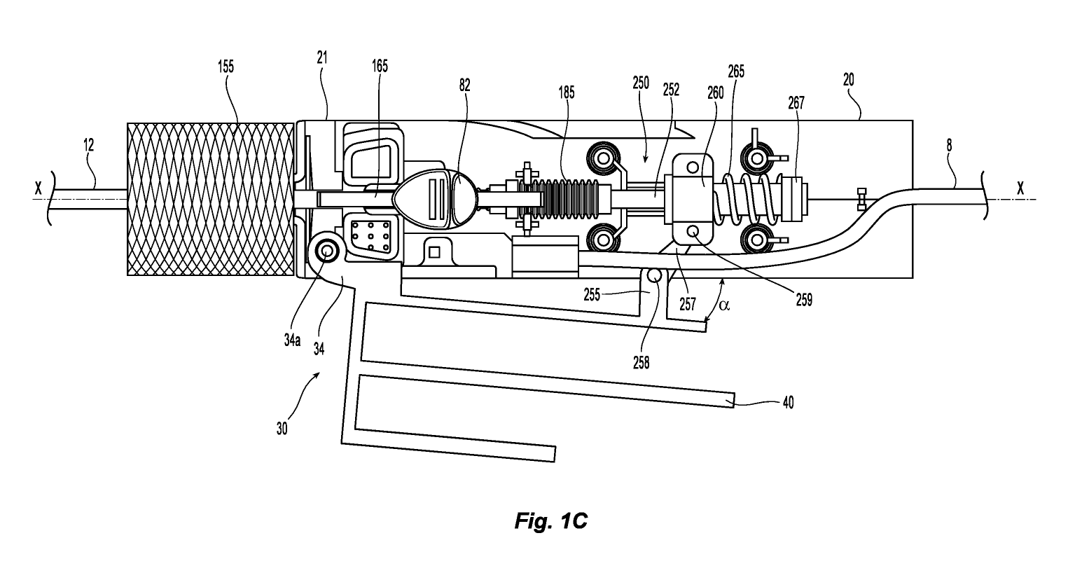

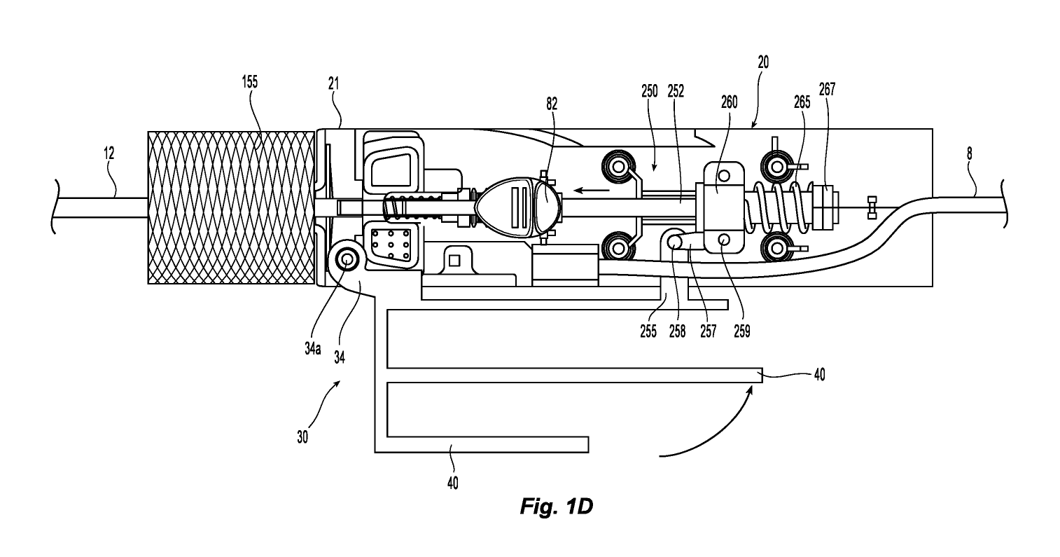

Referring initially to FIGS. 1A-1D, an in-line forceps 10 for use in connection with endoscopic surgical procedures is disclosed. The forceps 10 includes a housing 20 having a shaft 12 that extends therefrom which defines a longitudinal axis “X-X” therethrough. Housing 20 houses the internal working components of forceps 10. An end effector assembly 150 is disposed at a distal end 14 of the shaft 12 and includes first and second jaw members 110 and 120, respectively. A handle assembly 30 is operably coupled to the housing 20 and moveable relative thereto to actuate the jaw members 110 and 120 as explained in further detail below. A trigger assembly 80 is operable coupled to the housing 20 and is configured to advance a knife 175 between the jaw members 110 and 120 to cut tissue disposed therebetween as explained in more detail below. Forceps 10 may also be configured to rotate as explained in more detail below.

The distal end 14 of the shaft 12 is configured to mechanically engage end effector assembly 150 and a proximal end 16 of the shaft 12 is configured to mechanically engage housing 20. A cable 8 connects the forceps 10 to an energy source, e.g., a generator “G”, or other suitable power source, although forceps 10 may alternatively be configured as a battery-powered device. Cable 8 includes one or more wires (not shown) of sufficient length that extend through shaft 12 to provide energy to tissue-contacting surfaces 112, 122 (FIG. 1F) of jaw members 110, 120, respectively. A jaw member activation switch 200 is provided on the housing 20 and is configured to allow selective application of electrosurgical energy to the jaw members 110, 120. Switch 200 may work in electrical cooperation with a foot switch (not shown) to facilitate electrical application of the forceps 10. In addition and as explained below, switch 200 may also be part of an electrical safety circuit (not shown) to avoid unintended bipolar and/or monopolar activation.

Handle assembly 30 connects to a drive assembly 250 (FIGS. 1B-1D) which, together, mechanically cooperate to impart movement of one or both of the jaw members 110 and 120 between a first, open position wherein the jaw members 110 and 120 are disposed in spaced-apart relation relative to one another for manipulating tissue and a second, approximated position wherein the jaw members 110 and 120 cooperate to grasp tissue therebetween. As shown in FIG. 1A, handle assembly 30 includes a moveable handle 40 which is normally biased in a spaced-apart orientation relative to housing 20 and, correspondingly, jaw members 110, 120 are disposed in the first, open position. One or more commonly known biasing mechanisms may be employed for this purpose, e.g., a spring. Moveable handle 40 is actuatable from the initial, spaced-apart orientation to a compressed position corresponding to the approximated position of jaw members 110, 120.

A rotating assembly (not shown) may be included that is rotatable in either direction about longitudinal axis “X-X” to rotate end effector assembly 150 (i.e., jaw members 110 and 120) about longitudinal axis “X-X.” As explained in more detail below, a monopolar element 151 is moveably or slidingly housed within jaw member 120 and is selectively extendable therefrom. The monopolar element 151 rotates with the end effector assembly 150 to allow fine dissection of tissue.

Forceps 10 may also include a ratchet assembly (not shown) configured to selectively lock the jaw members 110 and 120 relative to one another at various points between the open and approximated positions. The ratchet assembly may include graduations or other visual markings to enable the user to easily and quickly ascertain and control the amount of closure force desired between the jaw members 110 and 120.

In some embodiments, one of the jaw members, e.g., 120, may include one or more stop members (not shown) disposed on an inner facing surface of the electrically conductive sealing surface 122 (and/or sealing surface 112 on jaw member 110). The stop member(s) is designed to facilitate gripping and manipulation of tissue and to define a gap distance between opposing jaw members 110 and 120 when approximated to promote quality tissue sealing. In embodiments, the gap distance “g” is within the range of about 0.001 inches (about 0.03 millimeters) to about 0.006 inches (about 0.016 millimeters).

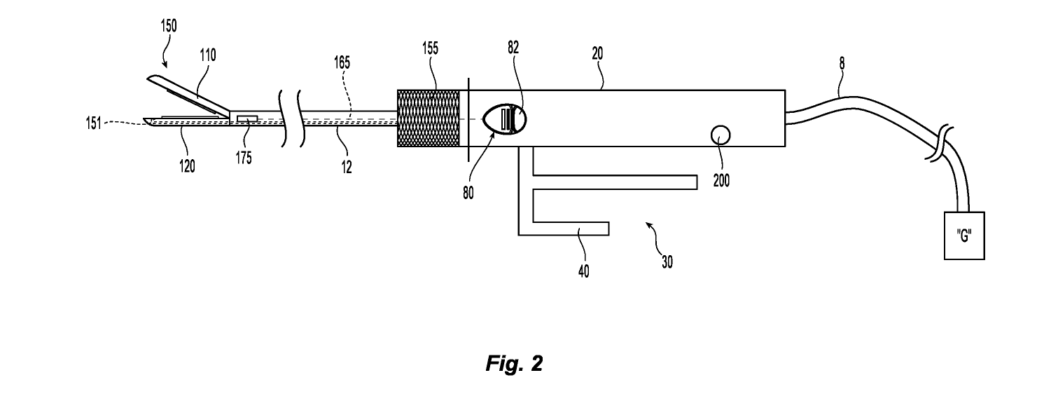

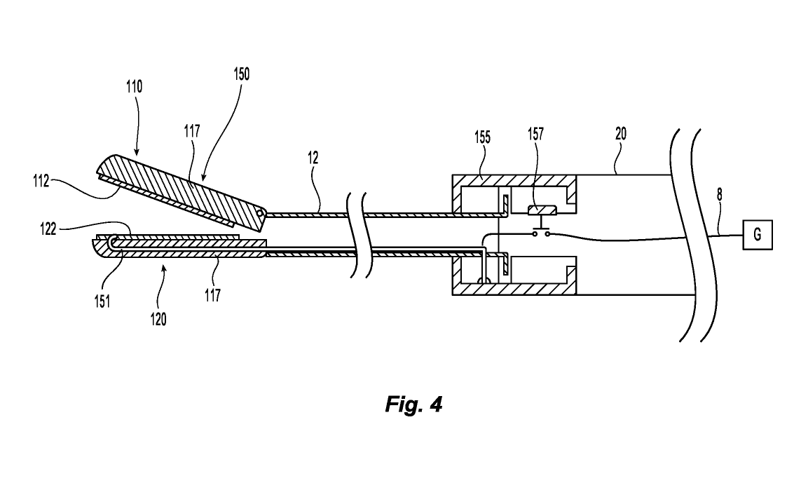

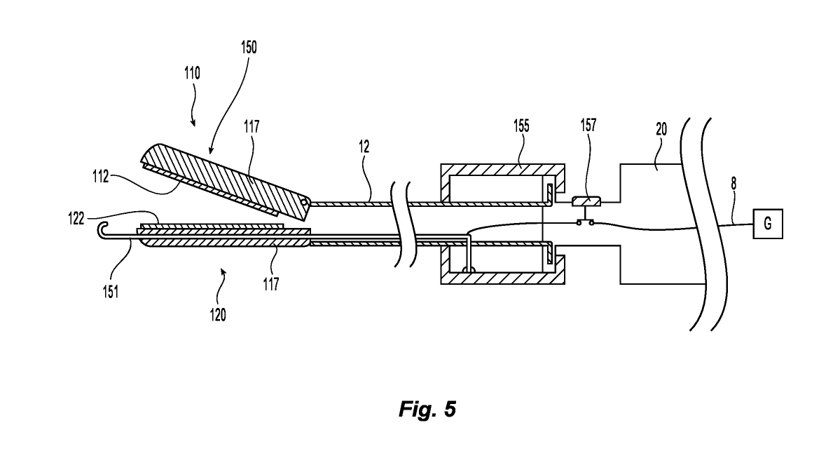

With reference to FIGS. 4 and 5, an enlarged view of the end effector assembly 150 is shown. Each jaw member 110, 120 of end effector assembly 150 includes an outer insulative jaw housing 117 and a tissue-contacting plate 112, 122, respectively. Further, one of the jaw members, e.g., jaw member 120, may include an energy-based cutting member (not shown) disposed thereon, or a channel (not shown in detail) for allowing a knife 175 to pass therethrough (See FIG. 2). Trigger 82 of the trigger assembly 80 is operably coupled to a knife rod 165 and is selectively translatable to advance the knife 175 through the knife channel (not shown) defined within one or both of jaw members 110, 120 to cut tissue disposed therebetween. Knife 175 may include a sharpened distal end for mechanical cutting, or may be selectively energizable, e.g., via one or more wires connected to the generator “G” for electro-mechanically cutting tissue.

In embodiments having a selectively energizable knife 175, the knife 175 may be coupled to the trigger 82 and generator “G” (FIG. 1A) such that energy, e.g., electrosurgical energy, may be selectively supplied to the knife 175 and conducted through tissue disposed between jaw members 110, 120 when the trigger is advanced. The knife channel (not shown) may be insulated in this instance. In addition, the knife 175 may alternatively be configured to conduct any suitable energy to the knife 175 to facilitate tissue cutting e.g., ultrasonic, optical, resistive, etc. Knife 175 may be biased in proximal-most position by one or more springs 185 (See FIG. 1C).

As mentioned above, the tissue-contacting plates 112, 122 are coupled to the activation switch 200 and generator “G” (or other suitable source of energy) via wires extending from cable 8 such that, in a first mode of operation, electrosurgical energy may be selectively supplied to tissue-contacting plate 112 and/or tissue-contacting plate 122 and through tissue disposed therebetween. In embodiments, e.g., a forceps 10 with an energy-based cutting member (not shown) disposed on one or both of the tissue-contacting plates 112, 122, the activation switch 200 may simultaneously seal and cut tissue or, alternatively, seal then cut tissue in a seal and cut cycle.

As best illustrated in FIGS. 1B-1D and as mentioned above, movable handle 40 mechanically couples to the housing 20 and is movable relative to the housing 20 to affect movement of the jaw members 110 and 120 from the open or spaced-apart configuration to the second, approximated position about tissue. While the drawings depict a device with one movable handle 40, more than one movable handle 40 may also be attached to the housing 20.

Movable handle 40 may be configured to extend downwardly at an angle alpha (a) relative to the longitudinal axis “X-X” defined through the housing 20. Manufacturing the movable handle 40 to extend in this fashion facilitates and enhances gripping and manipulation of the forceps 10 during operating conditions, however, linear or other actuation schema may be employed. The angle (a) of the movable handle 40 may be adjustable to allow different users to essentially “customize” the movable handle 40 for a particular use or for a particularly-sized hand. Alternatively, different forceps 10 may be manufactured with different pre-fixed angles (a) for use with specific surgical procedures, for particular hand sizes (e.g., small, medium and large) and/or for other surgical purposes. For example, larger forceps 10 may require longer hand strokes or greater angles. The angle (a) of the movable handle 40 may range from about zero degrees to about thirty-five degrees.

As best seen in FIGS. 1B-1D, the distal end 34 of movable handle 40 is selectively moveable about a pivot pin 34a attached to a distal end 21 of the housing 20. Movement of the moveable handle 40 relative to the housing 20 imparts movement of the drive assembly 250 which, in turn, moves the jaw members 110 and 120 relative to one another from the open position to the approximated position. Drive assembly 250 includes a flange 255 extending from handle 40 at a proximal end thereof that couples with a cam link 257 about a pivot 258. Cam link 257, in turn, couples to a collar 260 that rides atop a drive shaft 252 that operably engages the jaw members 110 and 120 at a distal end thereof such that translation of the drive shaft 252 actuates the jaw members 110 and 120 between the first, open position and the second, approximated position. Cam link 257 couples to collar 260 via a pivot 259 such that movement of handle 40 relative to housing 20 (e.g., towards housing 20) biases the collar 260 and a drive spring 265 against a drive stop 267 and translates the drive shaft 252 to force the jaw members 110 and 120 to the second, approximated position. The drive spring 265 regulates the compressive force of the jaw members 110 and 120 against tissue grasped therebetween. Upon release of the handle 40, the drive collar 260 is forced distally by the drive spring 265 such that the drive shaft 252 is translated in the opposite direction to return the jaw members 110 and 120 to the first, spaced apart position and the handle 40 is returned to its original, spaced-apart orientation relative to the housing 40.

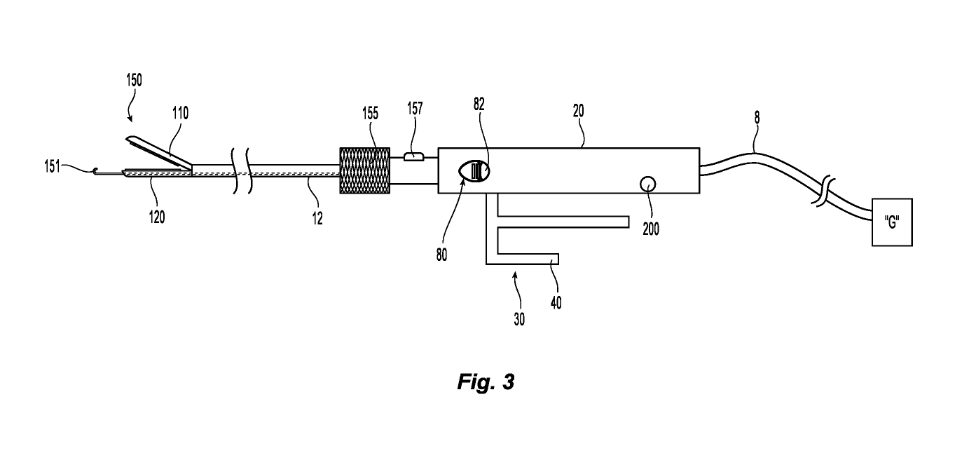

With reference now to FIGS. 2, 3, 4, and 5, monopolar element 151 is slidably disposed within the end effector assembly 150 and is movable between a retracted position (FIGS. 2 and 4) and an extended configuration (FIGS. 3 and 5). The forceps 10 includes an actuating sleeve 155 slidably mounted to the housing 20 and about the shaft 12. Actuating sleeve 155 is operably connected to the monopolar element 151 and movable between a first position (FIG. 4) wherein the monopolar element 151 is disposed in a retracted or housed configuration and one or more extended positions (FIGS. 3 and 5) wherein the monopolar element 151 is in an extended configuration relative to a distal end of jaw member 120. While the actuating sleeve 155 is shown on a distal portion of housing 20, the actuating sleeve 155 may be disposed anywhere along the housing 20, including but not limited to, a more proximal portion or at a proximal end thereof.

Actuating sleeve 155 may be movable relative to the housing 20 in any suitable manner. While actuating sleeve 155 is shown as being slidably movable relative to the housing 20, actuating sleeve 155 may also move in a screw-fit manner such that controllable, rotational motion of actuating sleeve 155 causes longitudinal movement as well. In such an embodiment, the actuating sleeve 155 is rotatable along a thread (not shown) and urges the monopolar element 151 distally. Any ratio of threads per inch is envisioned to promote advancement of the monopolar element 151.

In some embodiments, actuating sleeve 155 may alternatively or additionally be rotatable to rotate the end effector 150 about axis “X-X”. In this instance, a switch or toggle element (not shown) may be utilized to switch rotation of the actuating sleeve 155 between modes, e.g., rotational mode to rotate the end effector 150 and extension mode to advance or extend the monopolar element 151. Other suitable mechanical or electrical elements or features (not shown) may need to be implemented to accomplish this purpose. In another embodiment, rotation of the actuating sleeve 155 rotates the end effector assembly 150 and extension of the actuating sleeve 155 extends the monopolar element 151.

Actuating sleeve 155 may take any desired shape such as, but not limited to, a cylinder, a toroid, a cone, a trapezoid, or any other suitable shape. Actuating sleeve 155 may optionally include a frictional surface for enhancing grasping ability.

As shown in FIGS. 3-5, actuating sleeve 155 covers a monopolar activation switch 157 when in a first, retracted position (See FIG. 2) and exposes the monopolar activation switch 157 when moved to a second, extended position (See FIG. 3). In one embodiment, the activation sleeve 155 may be configured to allow actuation of switch 157 as soon as the user moves the actuating sleeve 155 enough to expose switch 157, e.g., at some point between the first and second positions of the actuating sleeve 155. In other embodiments, suitable safety mechanisms or features (explained below) are employed to prevent premature activation of the monopolar element 151 or simultaneous activation of the jaw members 110 and 120 and the monopolar element 151.

For example, in one embodiment, the actuating sleeve 155 must be fully extended to the second position to allow activation of switch 157. In this instance, one or more mechanical or electrical cut-off mechanisms may be employed for this purpose, e.g., actuators, sensors, safety switches, etc. (not shown). Thus, unless the user fully extends the actuating sleeve 155 (and, hence, the monopolar element 151) distally, electrosurgical energy cannot be supplied to the monopolar element 151, even if the switch 157 is accessible (i.e., electrical activation of switch 157 is a function of the position of actuating sleeve 155).

In other embodiments, the forceps 10 may be configured to prevent electrosurgical energy from flowing to the first and second jaw members 110 and 120 unless the actuating sleeve 155 is disposed in the first position. In this case, as soon as actuating sleeve 155 is moved toward the second position (e.g., past a sensor (not shown)), bipolar electrosurgical energy is no longer available to the jaw members 110 and 120 even despite possible actuation of switch 200.

In other embodiments, the actuating sleeve 155 (or the position thereof) prevents electrosurgical energy from flowing to the first and second jaw members 110 and 120 when the actuating sleeve 155 is disposed in the second position. In this case, bipolar energy is allowed to flow to the jaw members 110 and 120 up until the monopolar element 151 is fully extended and/or otherwise ready for use. Similar to the embodiments described above, this may be accomplished using one or more electrical switches, sensors, buttons, or the like that are configured to act as a cutoff between the generator “G” and the end effector 150.

In some embodiments, the safety mechanism or safety features include mechanical or electromechanical contacts operably associated with the actuating sleeve 155 that close or complete a circuit when the actuating sleeve 155 or the monopolar element 151 are disposed in a certain position. One or more sensors (not shown) may also be utilized for this purpose, e.g., a sensor that senses when the actuating sleeve 155 or monopolar element 151 are disposed in a certain position, e.g., an optical sensor, a proximity sensor, etc.

The forceps 10 may also be configured to allow simultaneous activation of the monopolar element 151 and cut off of bipolar energy to the end effector 150 and vice versa, in other words, only one switch 157 or 200 can be activated at a time. A sensor, circuit or mechanical contact (not shown) may also be utilized for this purpose.

In some embodiments, a portion of the end effector 150 may be used as a return for monopolar energy from the monopolar element 151. For example, either or both of the first and second jaw members 110 and 120 may be used as a return or partial return when the monopolar element 151 is activated. In other embodiments, monopolar energy is at least partially transmitted to an external electrode, such as, but not limited to, a return pad (not shown). Both an external electrode and one or more portions of the end effector 150 may also be used as the electrical return for the monopolar element 151 during the same activation.

In some embodiments, the monopolar element 151 is configured having a hook-like distal end. In other embodiments, the monopolar element 151 may be of any suitable geometry depending upon a particular surgical purpose, e.g., loop, partial loop, straight, tapered, flared, ball, needle, etc.

A method for performing a surgical procedure is also disclosed and includes sliding the actuating sleeve 155 from the first position toward the second position, thereby moving the monopolar element 151 from the retracted configuration toward the extended configuration and simultaneously exposing the monopolar activation switch 157. The method further includes positioning the monopolar element 151 proximate a desired tissue site and applying electrosurgical energy to tissue through the monopolar element 151 by activating the switch 157 to treat tissue.

Prior to or after activation of the monopolar element 151, the method may include advancing the jaw members 110 and 120 of the end effector assembly 150 to engage and approximate tissue therebetween. The method may also include activating the generator “G” to provide electrosurgical energy to the jaw members 110 and 120 to treat tissue in a bipolar manner to create a tissue seal or otherwise treat tissue, e.g., coagulate tissue. The method may also include actuating a knife 175 between the jaw members 110 and 120 to cut tissue disposed therebetween. Actuation of the knife 175 may occur prior to bipolar activation, during bipolar activation or after bipolar activation. Moreover, a step of the method may include deploying the knife 175 to cut tissue treated by the monopolar element 151.

The various embodiments disclosed herein may also be configured to work with robotic surgical systems and what is commonly referred to as “Telesurgery”. Such systems employ various robotic elements to assist the surgeon in the operating room and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely controls the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of the herein described forceps (e.g., end effectors, suction systems, knifes, etc.) which may complement the use of one or more of the embodiments described herein. The movement of the master handles may be scaled so that the working ends have a corresponding movement that is different, smaller, or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, suction strength/pressure drop, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

The master handles may be configured to operate actuating sleeve 155 to deploy the monopolar element 151 from the end effector 150. Alternatively, actuating sleeve 155 may configured to be actuated via one or more electro-mechanical motors between the first and second positions.

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The embodiments described with reference to the attached drawings are presented only to demonstrate certain examples of the disclosure. Other elements, steps, methods, and techniques that are insubstantially different from those described above and/or in the appended claims are also intended to be within the scope of the disclosure.

What is claimed is:

1. An electrosurgical instrument, comprising:
a housing having a shaft that extends therefrom;
an end effector assembly coupled to a distal end of the shaft, the end effector assembly including a monopolar element slidably disposed therein and configured to move between a retracted position and an extended configuration;
a monopolar activation switch disposed within the housing and configured to supply energy to the monopolar element upon actuation thereof; and
an actuating sleeve movable relative to the housing between a first position wherein the monopolar element is disposed in the retracted configuration and actuation of the monopolar activation switch is impeded and a second position wherein the monopolar element is disposed in the extended configuration and activation of the monopolar activation switch is unimpeded.

2. The electrosurgical instrument of claim 1, wherein the actuation sleeve covers the monopolar activation switch when disposed in the first position.

3. The electrosurgical instrument of claim 1, wherein the actuation sleeve exposes the monopolar activation switch for actuation thereof when disposed in the second position.

4. The electrosurgical instrument of claim 1, wherein the end effector assembly includes first and second jaw members, at least one of the first and second jaw members movable relative to the other between a spaced-apart position and an approximated position for grasping tissue therebetween, each jaw member including an electrically-conductive tissue-contacting surface adapted to connect to a source of electrosurgical energy to treat tissue grasped between the jaw members.

5. The electrosurgical instrument of claim 1, wherein electrosurgical energy is prevented from flowing to the monopolar element unless the actuating sleeve is proximate the second position.

6. The electrosurgical instrument of claim 1, wherein electrosurgical energy is prevented from flowing to the monopolar element unless the actuating sleeve is disposed in the second position.

7. The electrosurgical instrument of claim 4, wherein electrosurgical energy is prevented from flowing to the first and second jaw members unless the actuating sleeve is proximate the first position.

8. The electrosurgical instrument of claim 4, wherein electrosurgical energy is prevented from flowing to the first and second jaw members unless the actuating sleeve is disposed in the first position.

9. The electrosurgical instrument of claim 4, wherein electrosurgical energy is prevented from flowing to the first and second jaw members when the actuating sleeve is disposed proximate the second position.

10. The electrosurgical instrument of claim 1, wherein the configuration of the monopolar element is selected from the group consisting of hook, loop, flat blade, partial loop, straight, tapered, flared, ball, and needle.

11. The electrosurgical instrument of claim 1, further comprising an activation switch disposed on the housing and configured to supply electrosurgical energy to the end effector assembly.

12. The electrosurgical instrument of claim 11, wherein movement of the actuating sleeve from the first to second positions simultaneously places the monopolar activation switch in-circuit for selective energization of the monopolar element and places the activation switch out-of-circuit to impede the supply of electrosurgical energy to the end effector assembly.

13. The electrosurgical instrument of claim 4, wherein either or both of the first and second jaw members may be used as a monopolar return path when the monopolar element is energized.

14. A method for treating tissue, comprising:
providing a surgical instrument including a housing having a shaft that extends therefrom and an end effector assembly coupled to a distal end of the shaft, the end effector assembly including a monopolar element slidably disposed therein;
moving an actuating sleeve relative to the housing between a first position wherein the monopolar element is disposed in a retracted configuration and actuation of a monopolar activation switch is impeded and a second position wherein the monopolar element is disposed in an extended configuration and activation of the monopolar activation switch is unimpeded; and
actuating the monopolar activation switch disposed within the housing to supply electrosurgical energy to the monopolar element.

15. A method for treating tissue according to claim 14, wherein the surgical instrument includes an activation switch configured to energize the end effector assembly for treating tissue in a bipolar manner and wherein movement of the actuating sleeve from the first to second positions simultaneously places the monopolar activation switch in-circuit for selective energization of the monopolar element and places the activation switch out-of-circuit to impede the supply of electrosurgical energy to the end effector assembly.

16. A method for performing a surgical procedure, comprising:
moving an actuating sleeve relative to a housing of a surgical instrument to simultaneously expose a monopolar activation switch and extend a monopolar element from an end effector assembly disposed at a distal end of a shaft extending from the housing; and
actuating the monopolar activation switch to energize the monopolar element to treat tissue.

17. A method for performing a surgical procedure according to claim 16, wherein the moving of the actuation sleeve also deactivates an activation switch configured to energize the end effector assembly.
